(19) United States
(12) Patent Application Publication
(10) Pub. No.: US 2021/0228299 A1
(43) Pub. Date: Jul. 29, 2021

(54) APPARATUS FOR FACILITATING DONNING A GARMENT

(71) Applicant: GloveMe Scandinavia AB, Nyköping (SE)

(72) Inventors: Håkan Johan Löfholm, Huddinge (SE); Taisto Kalevi Nyström, Nyköping (SE); Patrik Härnman, Linköping (SE)

(73) Assignee: GloveMe Scandinavia AB, Nyköping (SE)

(21) Appl. No.: 17/053,540

(22) PCT Filed: May 16, 2019
(86) PCT No.: PCT/SE2019/050446

§ 371 Date: Nov. 6, 2020

(30) Foreign Application Priority Data
May 31, 2018 (SE) 1850658-4

(51) Int. Cl. A61B 42/50; A61B 42/40

(52) U.S. Cl. CPC A61B 42/50; A41D 2400/44; A61B 42/40

(57) ABSTRACT

The present invention relates to an apparatus (800) arranged for facilitating donning a garment (20) comprising a cuff (22) on a part of a human body. The apparatus (800) comprises a fetching arrangement (50) arranged to fetch a package (30) including a garment (20) from a storage (40). The apparatus (800) further comprises a garment and package opening arrangement (60) arranged to open the package (30) and thereby the cuff (22) of the garment (20). Furthermore, the apparatus (800) comprises a cuff opening arrangement (600) arranged to receive the opened cuff (22) of the garment (20), and to further open the cuff (22). Thereby, facilitating donning the garment (52) on a part of a human body.

### TECHNICAL FIELD

The present invention relates to an apparatus arranged for facilitating donning a garment comprising a cuff on a part of a human body.

### BACKGROUND

In this document, the embodiments of the present invention are often described in relation to gloves, such as disposable and/or sterile gloves. However, the present invention is not limited to usage in connection with gloves. Instead, present invention may be generally utilized for donning essentially any type of garment on essentially any body part.

Disposable gloves are today used by employees in a number of different areas, such as e.g. in the health care sector and the food industry. Disposable gloves are typically worn to protect the hands of the user and/or to prevent spreading of bacteria and other agents. Due to stricter hygienic requirements and safety aspects, the use of disposable gloves is increasing and is likely to continue to increase in the future.

### SUMMARY

One disadvantage with using disposable gloves is that it may be difficult and time consuming to put them on, especially if the gloves are tight and/or the hands are damp. In addition, the risk of damaging a glove when it is being put on is high. These drawbacks may not be that severe for persons which only occasionally uses disposable gloves. However, a lot of persons working with disposable gloves need to regularly change gloves. For example, a nurse typically puts on a new pair of gloves for each new patient, and a person working behind a deli counter puts on a new pair of gloves for each new customer. Hence, in certain professions, many glove changes are performed per day per employee. If each of these glove changes is time consuming, there are major time losses and thereby costs associated with the changing of disposable gloves.

The use of disposable gloves in environments with strict hygienic requirements poses additional challenges related to contamination. When disposable gloves are put on manually, the user typically needs to touch the outside of the disposable gloves. Although care is exercised, it may be difficult to avoid touching areas of the disposable glove which is later going to come in contact with e.g. a patient or food. Hence, there is a high risk that disposable gloves are contaminated as they are being put on.

To avoid the above described disadvantages related to applying and changing disposable gloves, machines which automatically apply gloves on the hands of a user have been developed. This type of machines is hereafter referred to as glove donning machines. Typically, a conventional glove donning machine uses vacuum or suction to inflate the glove and allow the hand to be inserted into the glove. One disadvantage with using vacuum or suction is the noise produced by the vacuum pump. In addition, it is difficult to control the suction power such that the glove can be opened properly. Other types of known glove donning machines rely on complex movement of mechanical parts and requires advance mechanical structures which may be prone to malfunction.

Consequently, there is a need for an improved glove donning machine/apparatus.

An objective of embodiments of the present invention is to provide a solution which mitigates or solves the drawbacks and problems of prior art.

The above and fur her objectives are achieved by the subject matter of the independent claim. Further advantageous implementation forms of the present invention are defined by the dependent claims and other embodiments.

According to an aspect of the invention, the above mentioned and other objectives are achieved with an apparatus arranged for facilitating donning a garment comprising a cuff on a part of a human body, the garment initially being included in a package stored in a storage; characterized by
a fetching arrangement arranged for fetching the package from the storage, and for placing the package in a package opening position, the fetching arrangement including:
   an arm arranged movable between the storage and the package opening position;
   at least one holding means arranged on the arm for holding the package at a first end portion of the package being opposite to a second end portion where the cuff of the garment is to be extracted from the package;
a garment and package opening arrangement arranged for moving a first and a second side of the package away from each other in the package opening position, whereby the package and the cuff of the garment are opened; and
a cuff opening arrangement arranged for receiving the opened cuff of the garment, and for further opening the cuff to facilitate the donning of the garment.

An advantage with the apparatus according to the aspect is that the garment and package opening arrangement opens the cuff of the garment such that the cuff opening arrangement can easily be inserted into the cuff of the garment. Thereby, an automated expansion of the cuff of the garment can be achieved which is both simple and fast. Furthermore, the cuff of the garment can be opened without the user having to touch the outside of to garment, thereby reducing the risk of contamination.

According to an embodiment of the invention, the at least one holding means is further arranged for holding the package and the garment at the first end portion of the package, whereby the package and the garment are held essentially vertical in the package opening position with the second end portion facing away from the at least one holding means.

An advantage with this embodiment is that when the package and the garment are held essentially vertically in the package opening position, the cuff of the garment can open inside the package when the first and second sides of the package are separated by the garment and package opening arrangement as no gravitational force is pushing the sides of the garment together. When the first and second sides of the package are separated, an expansion space is provided within the package. The garment is thus hanging without any package sides acting on the sides of the garment within this expansion space. The garment then strives to reach its relaxed state. The cuff, for e.g. a glove or another garment including a cuff having a slightly thicker material and/or being slightly more rigid/stiff/less flexible than the rest of the glove/garment, will then strive to return to its relaxed state, i.e. to a state in which the cuff forms an opening into the garment.

According to an embodiment of the invention, the at least one holding means is further arranged for holding the package at the first end portion of the package, whereby the package and the garment are held essentially horizontally in the package opening position with the second end portion facing away from the at least one holding means.

An advantage with this embodiment is that the package and the garment in the package opening position is held in a position which is suitable for donning of he garment and/or is suitable for some cuff opening arrangements.

According to an embodiment of the invention, the at least one holding means include one or more in the group of:
at least one clamp,
at least two pressing fingers,
at least two rollers.

According to an embodiment of the invention, the fetching arrangement further includes at least one retrieving means arranged for retrieving the package from the storage before the at least one holding means grabs hold of at least the package at the first end portion.

An advantage with this embodiment is that the holding means can more easily get a hold of the package at the first end portion when the retrieving means are included in the fetching arrangement.

According to an embodiment of the invention, the at least one retrieving means include one or more in the group of:
at least one clamp,
at least one pressing fingers,
at least one roller,
at least one suction device,
at least one adhesive covered surface.

According to an embodiment of the invention, the arm is extendable and tiltable.

An advantage with this embodiment is that the arm can easily and exactly be moved between the storage and the package opening position.

According to an embodiment of the invention, the apparatus further includes a package end opener arranged for opening the package at the second end portion.

An advantage with this embodiment is that less force is required to move the first and the second sides of the package away from each other such that package and the cuff of the garment are opened. Thereby, the garment and package opening arrangement can more easily open the package and the cuff of the garment.

According to an embodiment of the invention, the package end opener includes one or more in the group of:
at least one cutting device;
at least one ripping device.

According to an embodiment of the invention, the garment and package opening arrangement includes a first and a second grasping device arranged for grasping the first and second sides of the package, respectively.

An advantage with this embodiment is that the first and the second grasping devices provides a simple and reliable way of grasping the first and second sides of the package, respectively, which facilitates opening of the package.

According to an embodiment of the invention, each one of the first and second grasping devices includes at least one in the group of:
at least one suction device,
at least one clamping device arranged for clamping one of the first and second sides of the package,
at least one clamping device arranged for clamping at least one protrusion arranged on one of the first and second sides of the package,
at least one adhesive surface device arranged for adhering to one of the first and second sides of the package.

According to an embodiment of the invention, the first and second grasping devices are arranged to be moved away from each other when they have grasped the first and second sides of the package, respectively, thereby opening the package.

According to an embodiment of the invention, when the package is opened by the first and second sides being moved away from each other, a space is created between the first and second sides of the package into which the garment can relax, whereby the cuff is opened.

An advantage with this embodiment is that by opening the package, the cuff of the garment is opened to the extent that the cuff opening arrangement can easily be inserted into the cuff of the garment.

According to an embodiment of the invention, the cuff opening arrangement is arranged for placing the further opened cuff in a donning position.

An advantage with this embodiment is that the cuff opening arrangement places the cuff of the garment in a position which is suitable for donning, which facilitates donning of the garment, e.g. sterile gloves, without touching the packages and/or garments.

According to an embodiment of the invention, the cuff opening arrangement is arranged for receiving the garment in the package opening position.

An advantage with this embodiment is that the cuff opening arrangement can easily be inserted into the garment placed in the package opening position to facilitate further opening of the garment.

According to an embodiment of the invention, the cuff opening arrangement comprises a first and a second cuff opening assembly, each of the first and second cuff opening assemblies comprising:
a first arm comprising a first proximal end and a first distal end, the first arm being rotatable around a first geometrical axis;
a second arm comprising a second proximal end and a second distal end, the second arm being rotatable around a second geometrical axis; wherein
the first and second arms are arranged to rotate around the first and second geometrical axes, respectively, between an initial position and an opening position, such that the first and second distal ends are at a first initial distance from each other in the initial position, and are separated to a first opening distance from each other in the opening position, when the first and second arms are rotated around the first and second geometrical axes, respectively, the first opening distance being greater than the first initial distance.

An advantage with this embodiment is that the cuff opening arrangement can expand the cuff of the garment using rotating arms. Thereby, an automated expansion of the cuff of the garment can be achieved which is both simple and fast. The use of rotating arms further allows the apparatus to be made compact as the arms can be moved around their own axes.

According to an embodiment of the invention,
the first and second geometrical axes are arranged with an angle to each other, the angle being selected such that the first and second distal ends are at the first initial distance from each other the initial position, and are separated to the first opening distance from each other in the opening position, when the first and second arms are rotated around the first and second geometrical axes, respectively.

An advantage with this embodiment is that the first and second distal ends can be rotated and separated from each other in one movement. Thereby, a large expansion of the cuff of the glove can be achieved in a simple and fast way.

According to an embodiment of the invention, the garment is still included within the package after the donning on the part of the human body has been performed.

An advantage with this embodiment is that the garment can be protected from e.g. contamination after the donning.

Further applications and advantages of the present invention will be apparent from the following detailed description.

### BRIEF DESCRIPTION OF THE DRAWINGS

The appended drawings are intended to clarify and explain different embodiments of the present invention, in which:

FIGS. 1a-e show different views of an apparatus according to an embodiment of the invention;

FIGS. 2a-b show different views of a glove in a package according to an embodiment of the invention;

FIGS. 3a-b show a glove donned on a hand according to an embodiment of the invention;

FIGS. 4a-d show a holding means according to various embodiments of the invention;

FIGS. 5a-b show a retrieving means according to various embodiments of the invention;

FIGS. 6a-d shows a package end opener according to various embodiments of the invention;

FIGS. 7a-d show a garment and package opening arrangement according to various embodiments of the invention;

FIGS. 8a-d show different views of a cuff opening arrangement according to an embodiment of the invention;

FIGS. 9a-d show side views of a cuff opening arrangement according to various embodiment of the invention;

FIGS. 10a-d show top views of a cuff opening arrangement according to an embodiment of the invention.

### DETAILED DESCRIPTION

The invention is related to an apparatus which is arranged to facilitate donning a garment comprising a cuff on a part of a human body, where the garment initially included in a package stored in a storage. The apparatus fetches the package including the garment from the storage, opens the package, and expands the cuff of the garment such that the part of the human body can easily be inserted into the garment. In this way, the donning of the garment can be automated and a user the apparatus can put on the garment without having to touch the outside of the garment. The garment may e.g. be a glove or a sock, and the apparatus may hence be arranged to facilitate donning a glove on a hand of a user, or a sock on a foot of a user. In the embodiments described herein, the garment is exemplified with a glove, and the apparatus therefore facilitates donning a glove on a hand of a user. However, the same functions and principles applies to an apparatus according to embodiments of the invention for donning other type of garments on a part of a human body, e.g. donning a sock on a foot of a user.

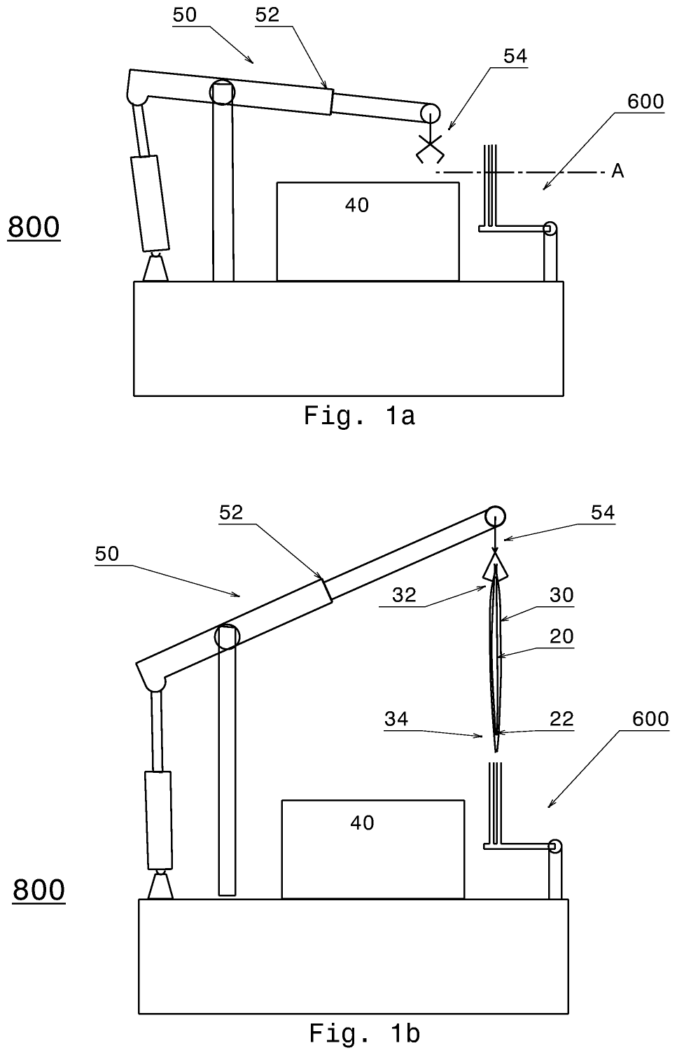

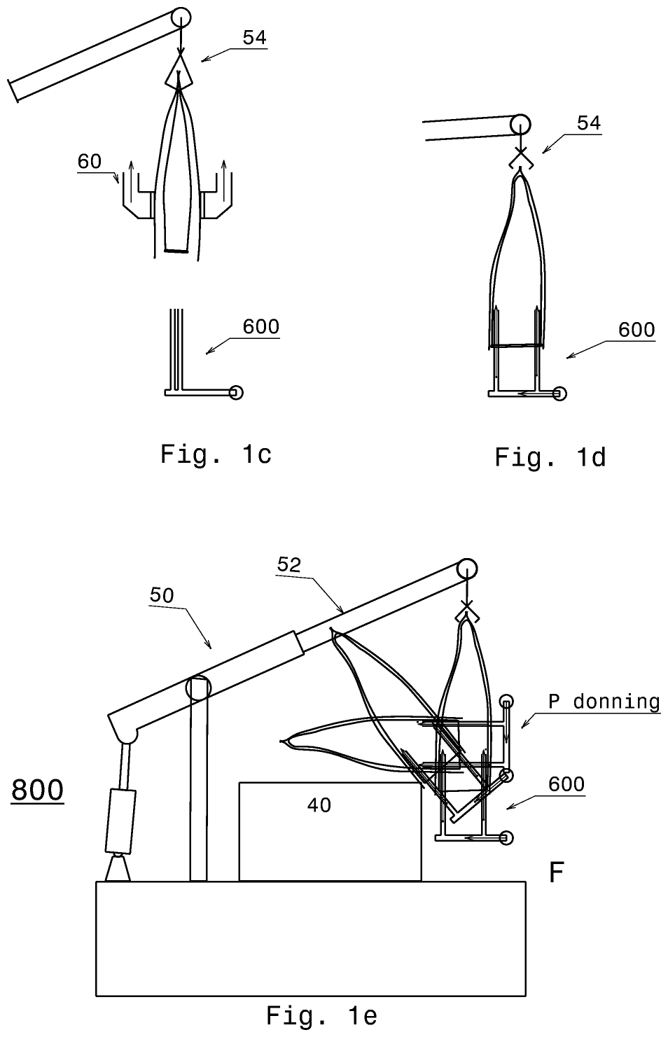

FIGS. 1a-e schematically illustrates an apparatus 800 according to an embodiment of the invention. In the embodiment shown in FIGS. 1a-e, the apparatus 800 is arranged to facilitate donning a glove 20 with a cuff 22 on a hand of a user. The apparatus 800 includes a storage 40 where the gloves 20 to be donned are stored. Each glove 20 is included in a package 30 and the packages 30 can be stored in a stack in the storage 40. Hence, the storage 40 can hold a stack of packages 30, e.g. with one package 30 at the top of the stack ready to be fetched for donning.

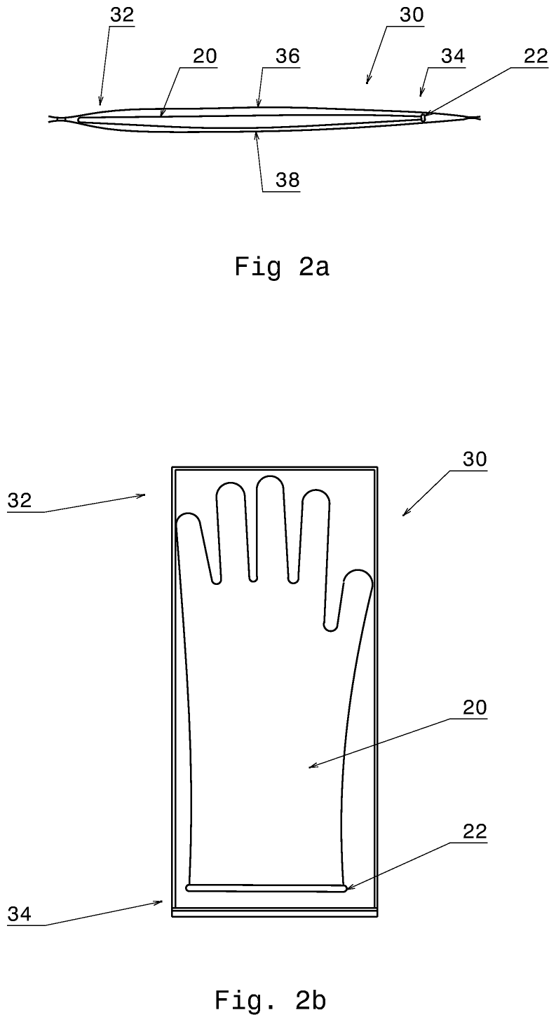

FIGS. 2a-b show a glove 20 in a package 30 according to an embodiment of the invention. As shown in FIGS. 2a-b, the glove 20 lays flat in its package 30 such that the fingertips of the glove 20 are at a first end portion 32 of the package 30 and the cuff 22 of the glove 20 is at an opposite second end portion 34 of the package 30. The second end portion 34 of the package 30 is the end through which the glove 20 is to be extracted from the package 30. Furthermore, one side of the glove 20 is facing a first side 36 of the package 30, while the opposite side of the glove 20 is facing a second side 38 of the package 30. If the glove 20 is to be used in a sterile environment, e.g. an operating room/theater, the glove 20 can be sterilely packaged inside the package 30.

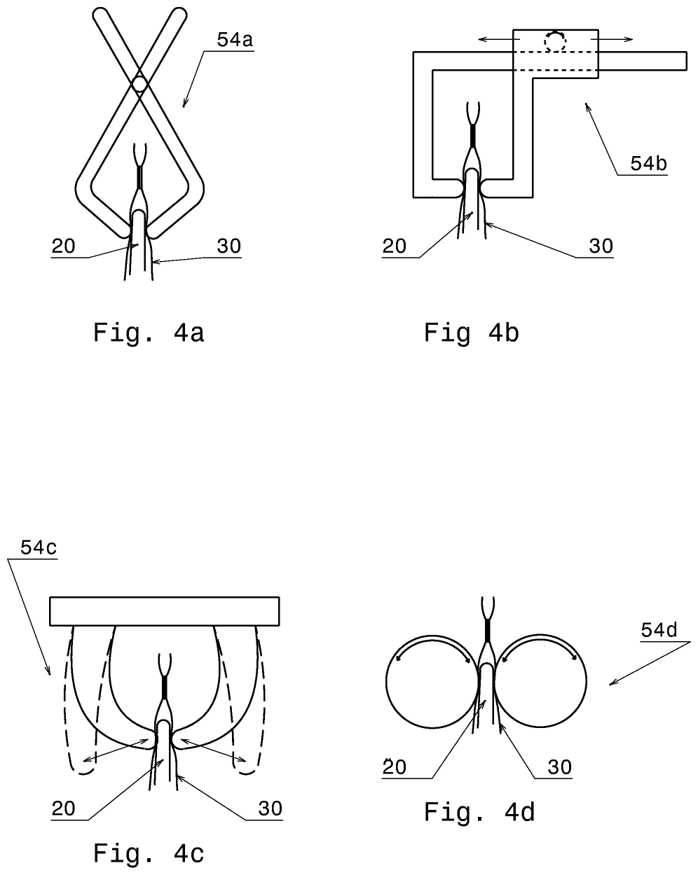

As illustrated e.g. in FIGS. 1a-e, the apparatus 800 according to the invention includes a fetching arrangement 50 arranged to fetch a package 30 from the storage 40 and to place the package 30 in a package opening position P_{open_package}. The fetching arrangement 50 may include an arm 52 arranged movable between the storage 40 and the package opening position P_{open_package} and at least one holding means 54 arranged on the arm 52. The holding means 54 is arranged to hold the package 30 such that the package 30 is moving with the arm 52. Hence, the fetching arrangement 50 uses the arm 52 to move the package 30, held by the holding means 54, from the storage 40 to the package opening position P_{open_package}. Various embodiments of the holding means 54 will be described below with reference to FIGS. 4a-d.

In the embodiments shown in FIGS. 1a-e, the arm 52 of the fetching arrangement 50 is extendable and tiltable. FIG. 1a shows the arm 52 in a retracted and tilted down position, where the fetching arrangement 50 can fetch a package 30 from the storage 40 using the holding means 54. The fetching action includes the holding means 54 grabbing hold of a package 30 from the storage 40, e.g. the package 30 at the top of a stack of packages 30 in the storage 40. The holding means 54 may, according to an embodiment, grab hold of the package 30 at the first end portion 32 such that the package 30, as well as the glove 20 inside the package 30, are firmly held by the holding means 54. When the package 30 has been fetch, i.e. grabbed by the holding means 54, the arm 52 is extended and tilted up to move the package 30 to the package opening position P_{open_package}. An embodiment of such a package opening position P_{open_package} is shown in FIG. 1b. In this package opening position P_{open_package}, the holding means 54 is arranged to hold the package 30 and the glove 20 at the first end portion 32 of the package 30 opposite to the second end portion 34 where the cuff 22 of the glove 20 is to be extracted from the package 30. Furthermore, the holding means 54 is arranged to hold the package 30 and the glove 20 essentially vertically in the package opening position P_{open_package} with the second end portion 34 facing away from the at least one holding means 54, as shown in FIG. 1b.

According to an embodiment, such a package opening position P_{open_package} may also be essentially horizontal, as described more in detail below.

Although an extendable and tiltable arm 52 is shown in the embodiments in FIGS. 1a-e, the arm 52 of the fetching arrangement 50 may in embodiment instead move in other ways without deviating from the scope of the invention. For example, the arm 52 may be arranged to move back and forth in a straight line from the storage to the package opening position P_{open_package}.

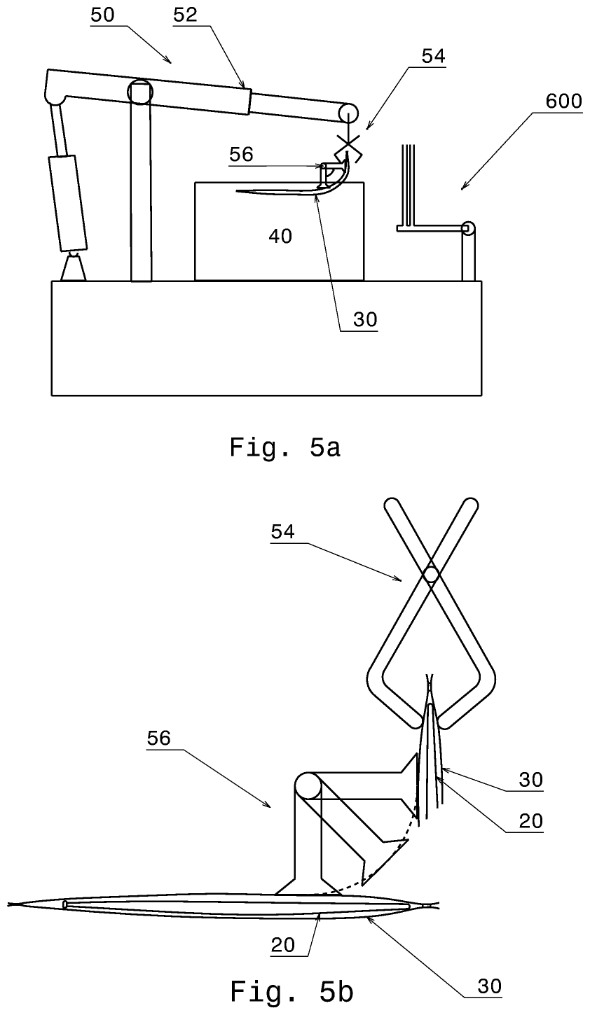

To allow the package 30 to easily be fetched from the storage 40, the fetching arrangement 50 may further include at least one retrieving means 56 arranged to retrieve the package 30 from the storage 40 before the at least one holding means 54 grabs hold of the package 30 and the glove 20 at the first end portion 32. The retrieving means 56 may e.g. attach to a package 30 in the storage and move the package 30 such that the first end portion 32 of the package 30 is outside the storage 40, where the holding means 54 can easily get a hold of the first end portion 32 of the package 30. An embodiment of the retrieving means 56 will be described below with reference to FIGS. 5a-b.

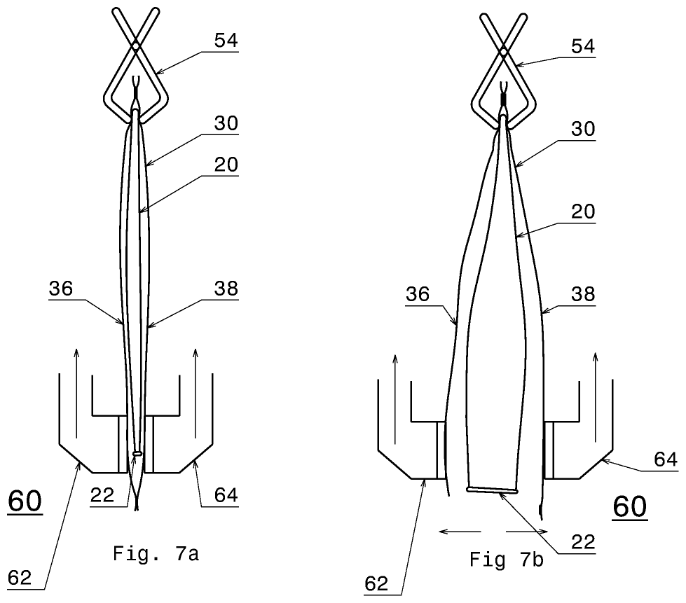

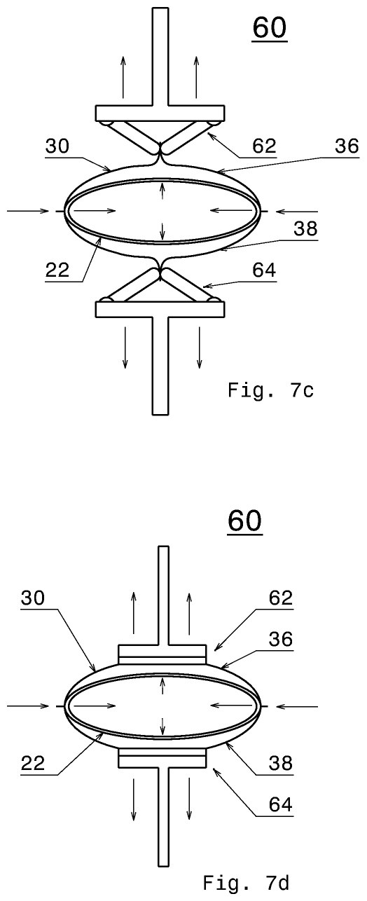

The apparatus 800 further includes a garment and package opening arrangement 60. The garment and package opening arrangement 60 is arranged to open a package 30 placed in the package opening position P_{open_package}. According to various embodiments of the invention, the garment and package opening arrangement 60 is arranged to move the first 36 and the second 38 side of the package 30 away from each other when the package 30 is in the package opening position P_{open_package}, such that the package 30 and the cuff 22 of the glove 20 are opened. FIG. 1c shows a garment and package opening arrangement 60 according to an embodiment of the invention. In the embodiment shown in FIG. 1c, garment and package opening arrangement 60 uses suction to attach to the first 36 and the second 38 sides of the package 30 and move them away from each other. As can be seen in FIG. 1c, when the first 36 and the second 38 side of the package 30 are moved away from each other a space is created between the first 36 and the second 38 sides of the package 30 into which the glove 20 can relax/expand, thereby opening the cuff 22 of the glove 20. Further embodiments of the garment and package opening arrangement 60, as well as details related to the function of the garment and package opening arrangement 60, will be described below with reference to FIGS. 7a-d.

The garment and package opening arrangement 60 opens the package 30 and thereby the cuff 22 of the glove 20. However, the opening of the cuff 22 of the glove 20 provided by the garment and package opening arrangement 60 may not be large enough for a hand to easily be inserted into the glove 20. Hence, the apparatus 800 includes a cuff opening arrangement 600 arranged to receive the cuff 22 of the glove 20 opened by the garment and package opening arrangement 60 and to further open the cuff 22 of the glove 20 to facilitate the donning of the glove 20. The cuff opening arrangement 600 can be arranged to receive the glove 20 in the package opening position P_{open_package}, where the package 30, or the package 30 and the glove 20, are held by the holding means 54. In various embodiments, the cuff opening arrangement 600 may e.g. include arms which are inserted into the glove 20 held in the the package opening position P_{open_package}. FIGS. 1a-e show such an embodiment. Once inserted into the glove 20, the arms of the cuff opening arrangement 600 are moved apart to further open the cuff 22 of the glove 20, as shown in FIG. 1d. The cuff 22 of the glove 20 may be opened to the extent that a hand can easily be inserted into the glove 20. When the cuff opening arrangement 600 has opened the cuff 22 of the glove 20 further, the package 30 and the glove 20 are held by the cuff opening arrangement 600 and the hold of the holding means 54 can be released, as shown in FIG. 1d.

To facilitate the donning of the glove 20, the cuff opening arrangement 600 may be arranged to place the package 30 and the glove 20 with the further opened cuff 22 in a donning position P_{donning}. As shown in FIG. 1e, this may, according to an embodiment, include turning at least a part of the cuff opening arrangement 600 such that the glove 20 is essentially horizontal with the cuff 22 of the glove 20 facing a front F of the apparatus 800. Thereby, a user can easily insert his or her hand into the glove 20 held open by the cuff opening arrangement 600.

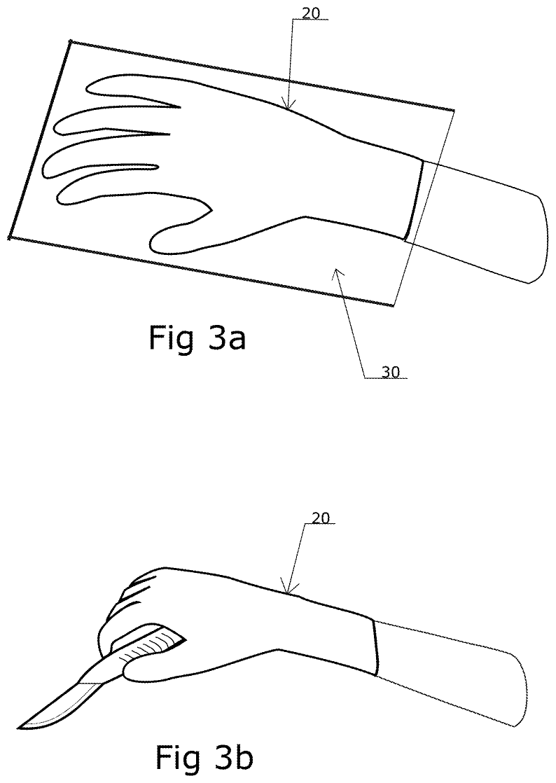

The glove 20 can be opened inside the package 30. Hence, the glove 20 may still be included within the package 30 after the donning on the hand has been performed. In this case, the user may after the donning be wearing the glove 20 covered by the package 30, as shown in FIG. 3a. In this way, the glove 20 is still protected by the package 30 and the package 30 can be removed just before the glove 20 is used. To keep the glove 20 inside the package 30 after the donning may be beneficial in a sterile environment such as e.g. an operating theater. For example, when preparing for an operation a surgeon can put on a pair of gloves 20 still covered by their respective packages 30. The packages 30 are then removed just before the operation is started, thereby minimizing the risk of contaminating the gloves 20. FIG. 3b shows a glove 20 donned on a hand after the removal of the package 30.

According to the embodiment shown in FIGS. 1a-e, the holding means 54 is arranged to hold the package 30 and the garment 20 essentially vertically in the package opening position P_{open_package}. However, as mentioned above, according to some embodiments of the invention, the holding means 54 may instead be arranged to hold the package 30 at the first end portion 32 of the package 30 such that the package 30 and the garment 20 are held essentially horizontally in the package opening position P_{open_package} with the second end portion 34 facing away from the at least one holding means 54. The holding means 54 may e.g. after fetching the package 30 from the storage 40 hold the package 30 in the donning position P_{donning}, shown in FIG. 1e. In other worlds, the package opening position P_{open_package} may in embodiments correspond to the donning position P_{donning}.

According to some embodiments, the holding means 54 may also be arranged to hold the package 30 at an angle in between a vertical position and a horizontal position in the package opening position P_{open_package}.

In embodiments where the package opening position Po_{open_package} is not essentially vertical, gravitational forces may act on at least one of the sides of the glove 20 such that there are forces acting on the sides of the glove 20 trying to hold the sides of the glove 20 together, thereby making it harder for the cuff 22 of the glove 20 to open when the package 30 is opened. Hence, in such embodiments the cuff 22 of the glove 20 may be arranged such that the cuff 22 is able to expand even when a gravitational force is acting on at least one of the sides of the glove 20. For example, the cuff 22 may be stiffer/more rigid, e.g. by being of thicker material and/or of less flexible material than the rest of the garment, in embodiments where the package opening position P_{open_package} is not essentially vertical compared to embodiments where the package opening position P_{open_package} is essentially vertical.

The holding means 54 may he implemented in various ways using various means/elements/devices such as e.g. clamps, pressing fingers, and/or rollers. FIGS. 4a-d show the holding means 54 according to some embodiments of the invention, where the holding means 54 is arranged to hold the package 30 and the garment 20 essentially vertically in the package opening position P_{open_package}. As shown in FIGS. 4a-d, the holding means 54 holds the package 30 at the first end portion 32 such that the package 30, as well as the glove 20 inside the package 30, is firmly held by the holding means 54. The holding means 54 shown FIGS. 4a-d may also be used in embodiments where the holding means 54 is arranged to hold the package 30 and the garment 20 essentially horizontally in the package opening position P_{open_package}. In this case, the holding means 54 may be arranged to hold both the package 30 and the garment 20, or to only hold the package 30.

FIGS. 4a-b show two different types of clamps 54a, 54b which can be used to implement the holding means 54. The clamp 54a shown in FIG. 4a is based on a scissor-like function, while the clamp 54b shown in FIG. 4b is based on a sliding motion. In FIG. 4c, the holding means 54 includes two pressing fingers 54c able to move from an open to a closed position e.g. using mechanical or pneumatic means. FIG. 4d shows an embodiment where the holding means 54 is implemented using two rollers 54d which grabs the package 30 and garment 20 by rolling onto the package 30 in opposite directions.

To summarize, the holding means 54 may include one or more in the group of at least one clamp 54a; 54b, at least two pressing fingers 54b, and at least two rollers 54c.

In a similar way as for the holding means 54, the retrieving means 56 may be implemented in various ways. In addition to the previously mentioned means, the retrieving means 56 may further use means such as suction devices or adhesive surfaces. FIGS. 5a-b show the retrieving means 56 according to an embodiment of the invention. In the embodiment shown in FIGS. 5a-b, the retrieving means 56 comprise at least one suction device. The suction device is arranged to move between a position in which the suction device may attach to a package 30 in the storage 40 to a position in which the suction device holds the package 30 such that the holding means 54 may easily grab hold of the first end portion 32 of the package 30, as shown in FIGS. 5a-b.

To summarize, the retrieving means 56 may include one or more in the group of at least one clamp 56a, at least one pressing fingers 56b, at least one roller 56c, at least one suction device 56d, and at least one adhesive covered surface 56e.

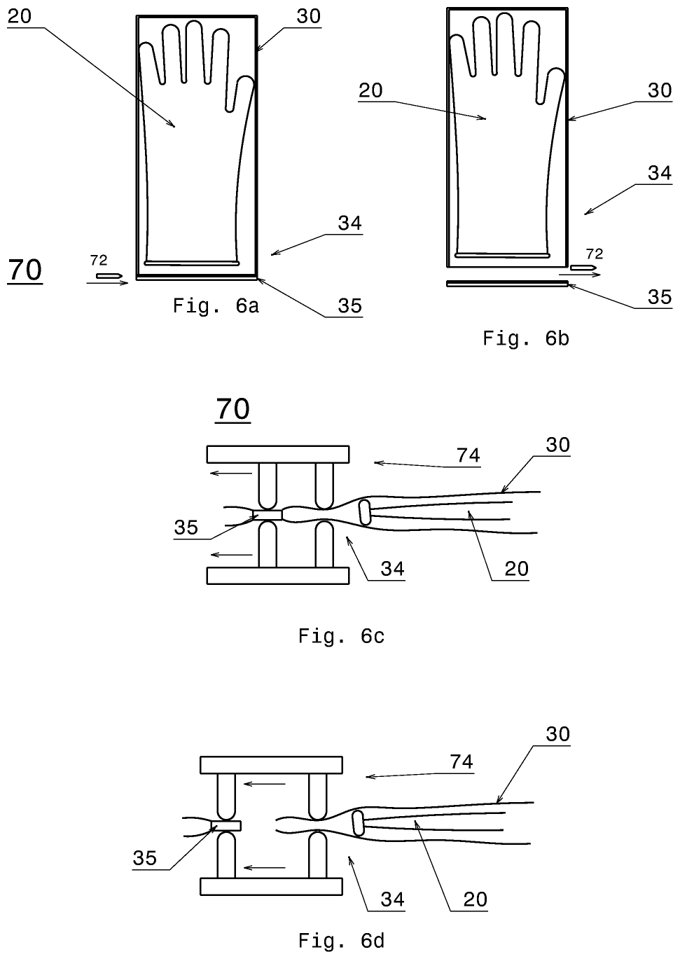

According to some embodiments of the invention, the second end portion 34 of the package 30 may be opened before the package 30, and there the cuff 22 of the glove 20, is opened by the garment and package opening arrangement 60. In such embodiments, the apparatus 800 further includes a package end opener 70 arranged to open the package 30 at the second end portion 34. The package end opener 70 may include one or more in the group of at least one cutting device 72 and at least one ripping device 74. FIGS. 6a-b shows a package end opener 70 using a cutting device 72 to open the package 30 at the second end portion 34. The cutting device 72 may e.g. be a knife or a laser moving along the package 30 at the second end portion 34 and thereby cutting off a sealed end 35 of the package 30, as shown in FIGS. 6a-b. FIGS. 6c-d show a package end opener 70 using a ripping device 74 to open the package 30 at the second end portion 34. The ripping device 74 may be arranged to rip of a sealed end 35 of the package 30 such that the package 30 is opened at the second end portion 34, as shown in FIGS. 6c-d.

As previously described, the garment and package opening arrangement 60 is arranged to open the package 30 and the cuff 22 of the glove 20 in the package opening position P_{open_package} by moving the first 36 and the second 38 side of the package 30 away from each other. The garment and package opening arrangement 60 may move the first 36 and the second 38 side the package 30 away from each other using grasping devices.

For example, the garment and package opening arrangement 60 may include a first 62 and a second 64 grasping device arranged for grasping the first 36 and second 38 sides of the package 30, respectively, as shown in FIG. 7a. The first 62 and second 64 grasping devices are arranged to be moved away from each other when they have grasped the first 36 and second 38 sides of the package 30, respectively, thereby opening the package 30. When the package 30 is opened by the first 36 and second 38 sides being moved away from each other, the glove 20 tries to reach its relaxed state in the package opening position P_{open_package} whereby the cuff 22 is opened, as shown in FIG. 7b.

In the embodiment shown in FIGS. 7a-b, the first 62 and second 64 grasping devices of the garment and package opening arrangement 60 are both suction devices. Air is sucked in through the first 62 and second 64 grasping devices thereby grasping the first 36 and second 38 sides of the package 30, respectively. However, the first 62 and second 64 grasping devices of the garment and package opening arrangement 60 may instead use other ways to grasp the first 36 and second 38 sides of the package 30. FIG. 7c shows an embodiment where she first 62 and second 64 grasping devices of the garment and package opening arrangement 60 are both clamping devices. In FIG. 7c, the first 62 and second 64 grasping devices clamp a protrusion on the first 36 and second 38 sides of the package 30, respectively. In embodiments, the first 62 and second 64 grasping devices may however instead clamp on directly to the first 36 and second 38 sides of the package 30, respectively. FIG. 7d shows an embodiment where the first 62 and second 64 grasping devices of the garment and package opening arrangement 60 are both adhesive surface devices. Each of the first 62 and second 64 grasping devices includes an adhesive surface which can attach to the first 36 and second 38 sides of the package 30, respectively. According to an embodiment, different grasping devices herein mentioned may be arranged on the first 36 and second 38 sides of the package 30.

For each one of the various embodiments used for the garment and package opening arrangement 60, when the first 62 and second 64 grasping devices have grasped the first 36 and second 38 sides of the package 30, respectively, the first 62 and second 64 grasping devices move away from each other. Thereby, the first 36 and second 38 sides of the package 30 move away from each other, creating an expansion space between the first 36 and second 38 sides of the package 30. The glove 20 relaxes/expands the space and the cuff 22 of the glove 20 forms an oval shape, as shown in FIGS. 7c-d. It should be noted that when the first 36 and second 38 sides of the package 30 are separated, the opening of the package 30 gets a more round/oval shape, as is shown in FIGS. 7c-d. This more round/oval shape helps forcing the cuff 22 of the glove 20 to open up, since the first and second sides of the garment being closest to the first 36 and second 38 sides of the package 30 tend to follow the first 36 and second 38 sides of the package 30, thereby being separated from each other, whereas the short end sides of the glove 20 are pushed slightly together, resulting in essentially oval shapes for both of the cuff 22 of the glove 20 and the package. These movements of the glove 20 and package 30 are indicated with arrows in FIGS. 7c-d.

Thus, to summarize, each one of the first 62 and second 64 grasping devices of the garment and package opening arrangement may include at least one in the group of:
at least one suction device,
at least one clamping device arranged for clamping one of the first 36 and second 38 sides of the package 30,
at least one clamping device arranged for clamping at least one protrusion arranged on one of the first 36 and second 38 sides of the package, and
at least one adhesive surface device arranged for adhering to one of the first 36 and second 38 sides of the package 30.

In the embodiment shown in FIGS. 1a-e, the apparatus 800 comprises one cuff opening arrangement 600. Hence, the apparatus 800 can be used for donning one glove 20 at a time. However, the apparatus 800 can comprise more than one cuff opening arrangement 600, e.g. two cuff opening arrangements 600. In this case, the apparatus 800 can be used for donning two gloves 20 at a time.

The herein disclosed embodiments of the apparatus 800 arranged to facilitate donning a garment 20 comprising a cuff 22 on a hand may include essentially any suitable cuff opening arrangement. Thus, essentially any cuff opening arrangement 600 being able to further open the cuff 22 such that donning of the garment is facilitated may be comprised in the apparatus 800. Such cuff opening arrangement 600 may for example utilize linear and/or rotational movement of one or more arms, causing one cuff 22 of the garment 20 to open such that a body part may be inserted into the garment, e.g. a hand being inserted into a glove. Also, essentially any other movement causing the cuff 22 to be further opened may be utilized. Below is one example of such a cuff opening arrangement 600 described in detail. However, the present invention is not limited to the below presented cuff opening arrangement 600. Instead, the present invention is generally applicable in combination with essentially any kind of cuff opening arrangement 600.

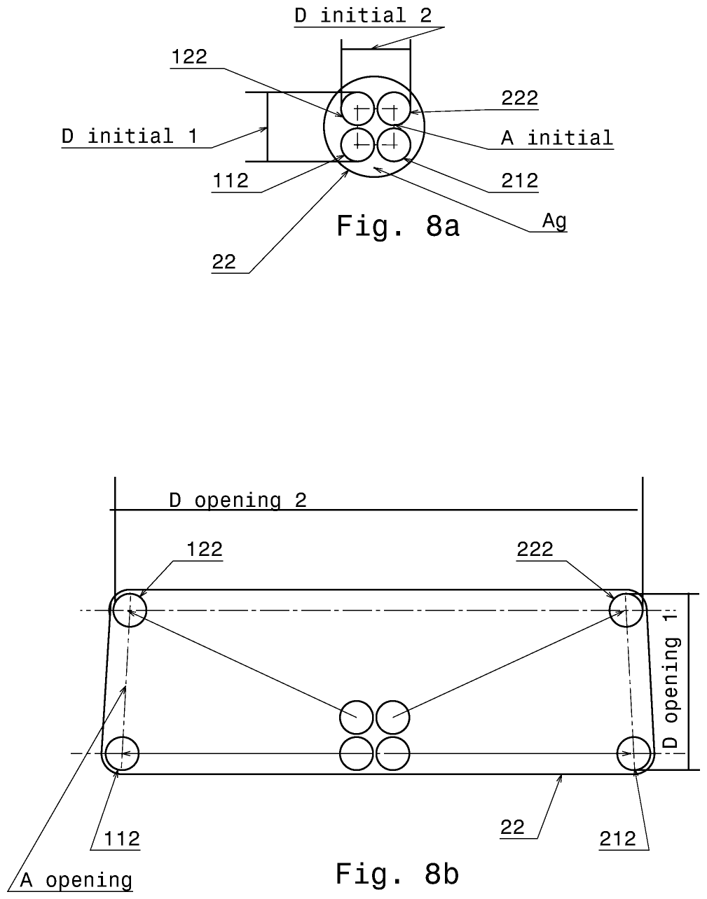

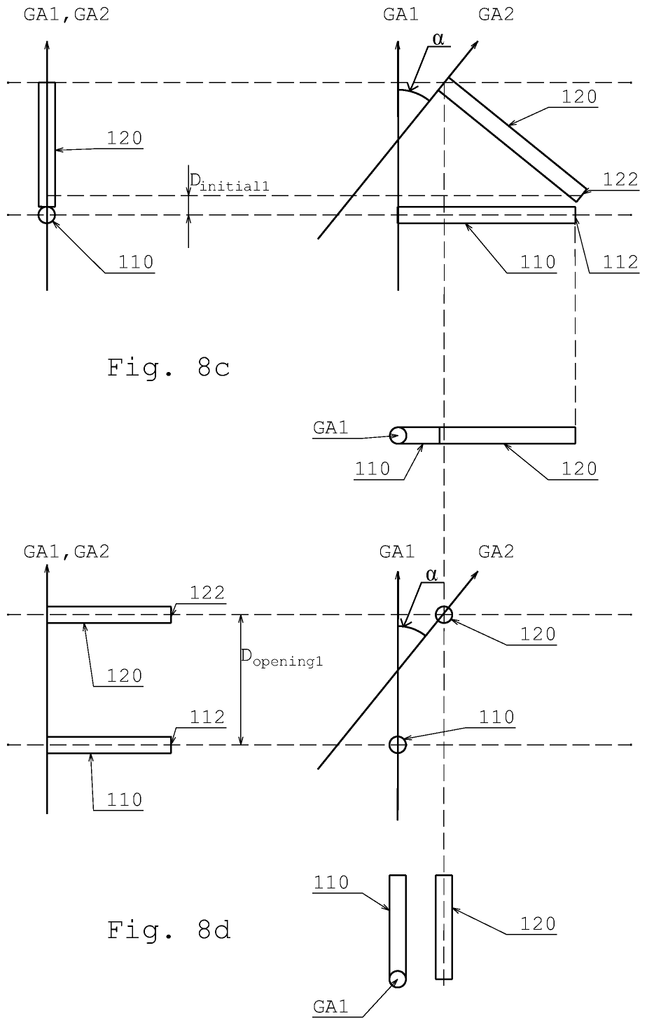

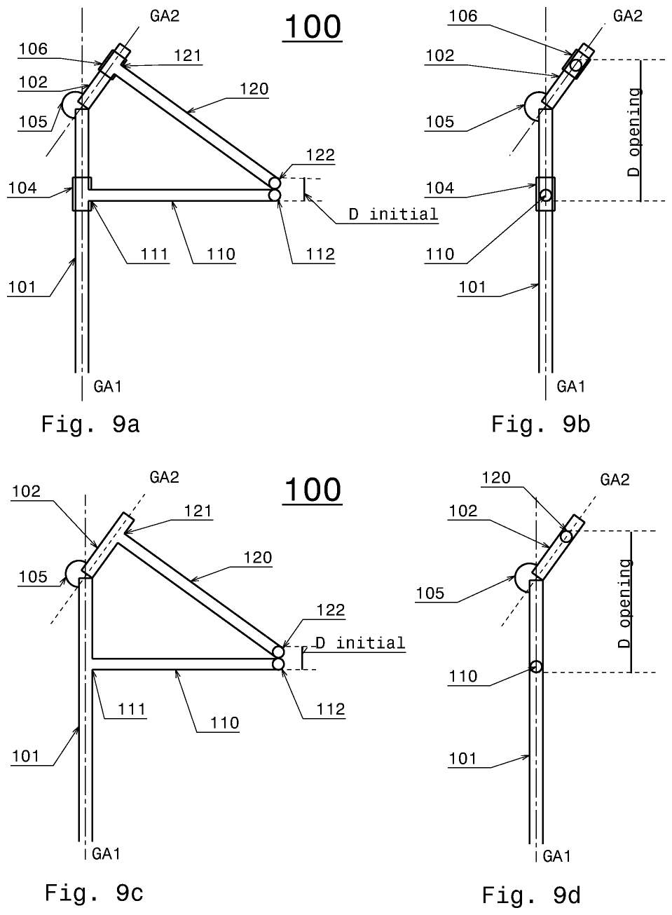

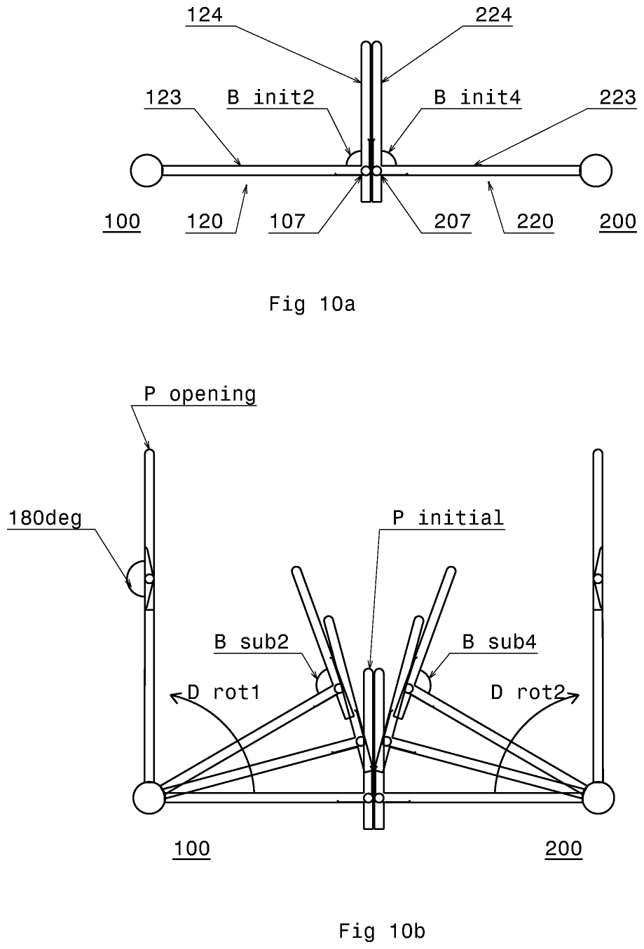

According to an embodiment of the invention each cuff opening arrangement 600 comprises a first 100 and a second 200 cuff opening assembly (shown in e.g. FIGS. 10a-b). Each of the first 100 and second 200 cuff opening assemblies comprises a first arm 110, 210 and a second arm 120, 220 (shown in e.g. FIGS. 9a-10d). The first 110, 210 and second 120, 220 arms of each of the first 100 and second 200 cuff opening assemblies are arranged to be inserted into a cuff 22 of a glove 20 and move apart such that the cuff 22 of the glove 20 is expanded, making the cuff 22 of the glove 20 bigger than it is in its relaxed state. As will now be described with reference to FIGS. 8a-b.

FIGS. 8a-b shows a cross section along the line A in FIG. 1a of the apparatus 800 and illustrate the movement of the first 100 and second 200 cuff opening assemblies from an initial position Pᵢₙᵢₜᵢₐₗ to an opening position P_{opening}. During the movement, a first arm 110 and a second arm 120 of the first cuff opening assembly 100, and a first arm 210 and a second arm 220 of the second cuff opening assembly 200, all move relative to each other. The first arms 110, 210 of the first 100 and second 200 cuff opening assemblies, respectively, comprises a first proximal end 111, 211 and a first distal end 112, 212, and is rotatable around a first geometrical shaft axis GA1, as illustrated in FIGS. 9a-d and 10a-b. In a similar way, the second arm 120, 220 of the first 100 and second 200 cuff opening assemblies, respectively, comprises a second proximal end 121, 221 and a second distal end 122, 222, and is rotatable around a second geometrical axis GA2. As FIGS. 8a-b shows a cross section of a part of the apparatus 800, only the first 112, 212 and the second 122, 222 distal ends of the first 100 and second 200 cuff opening assemblies, respectively, are shown in these figures. The other parts of the first 100 and second 200 cuff opening assemblies are shown in FIGS. 9a-d and 10a-b, as mentioned above.

The first 110, 210 and second 120, 220 arms are arranged to rotate around the first GA1 and second GA2 geometrical axes, respectively, between the initial position Pᵢₙᵢₜᵢₐₗ and the opening position P_{opening}. FIG. 8a shows the positions of the first 112, 212 and second 122, 222 distal ends relative to each other and relative to an opening of a glove 20 in the donning position P_{donning}, when the first 110, 210 and second 120, 220 arms are in the initial position Pᵢₙᵢₜᵢₐₗ. As shown in FIG. 8a, the first 112, 212 and second 122, 222 distal ends are at a first initial distance Dᵢₙᵢₜᵢₐₗ₁ from each other in the initial position Pᵢₙᵢₜᵢₐₗ. In addition, the first 112 and the second 122 distal ends of the first 110 and second 120 arms of the first cuff opening assembly 100 are at a second initial distance Dᵢₙᵢₜᵢₐₗ₂ from the first 212 and the second 222 distal ends of the first 210 and second 220 arms of the second cuff opening assembly 200, respectively, in the initial position Pᵢₙᵢₜᵢₐₗ. The first initial distance Dᵢₙᵢₜᵢₐₗ₁ and the second initial distance Dᵢₙᵢₜᵢₐₗ₂ are arranged to be small, e.g. close to zero, such that the first 112, 212 and second 122, 222 distal ends are close together in the initial position Pᵢₙᵢₜᵢₐₗ. Thereby, the first 112, 212 and second 122, 222 distal ends may easily be inserted into a cuff 22 of a glove 20 in the donning position P_{donning} when the cuff 22 of the glove 20 is in a relaxed, i.e. not expanded/unexpanded state. This is illustrated in FIG. 8a, which also shows an initial area Aᵢₙᵢₜᵢₐₗ and an area of an opening of the garment A_{g} in its relaxed state. The initial area Aᵢₙᵢₜᵢₐₗ is defined by the first 112 and second 122 distal ends of the first 110 and second 120 arms of the first cuff opening assembly 100 and the first 212 and second 222 distal ends of the first 210 and second 220 arms of the second cuff opening assembly 200, in the initial position Pᵢₙᵢₜᵢₐₗ. As shown in FIG. 8a, the initial area Aᵢₙᵢₜᵢₐₗ is smaller than the area of the opening of the glove A_{g} in its relaxed state.

When the first 110, 210 and second 120, 220 arms are rotated around the first GA1 and second GA2 geometrical axes, respectively, from the initial position Pᵢₙᵢₜᵢₐₗ towards the opening position P_{opening}, the first 112, 212 and second 122, 222 distal ends are separated from each other. The separation caused by the rotation takes place both horizontally and vertically, i.e. the first distal ends 112, 212 move away from the second distal ends 122, 222 of each one of the first 100 and second 200 cuff opening assembly, respectively, at the same time as the first 112 and second 122 distal ends of the first cuff opening assembly 100 move away from the first 212 and second 222 distal ends of the second cuff opening assembly 200, as indicated by the arrows in FIG. 8b. Furthermore, the first 112, 212 and second 122, 222 distal ends may be inserted into the cuff 22 of the glove 20 placed in the donning position P_{donning} when the first 110, 210 and second 120, 220 arms are rotated around the first GA1 and second GA2 geometrical axes, respectively, from the initial position Pᵢₙᵢₜᵢₐₗ to the opening position P_{opening}. In this way, the first 112, 212 and second 122, 222 distal ends are separated from each other inside the glove 20 and starts to expand the cuff 22 of the glove 20 during the rotation.

Further rotation of the first 110, 210 and second 120, 220 arms around the first GA1 and second GA2 geometrical axes, respectively, brings the first 110, 210 and second 120, 220 arms to the opening position P_{opening}. FIG. 8b shows the positions of the first 112, 212 and second 122, 222 distal ends relative to each other, when the first 110, 210 and second 120, 220 arms are in the opening position P_{opening}. As shown in FIG. 8b, the first 112, 212 and second 122, 222 distal ends of each one of the first 100 and second 200 cuff opening assembly, respectively, have been separated to a first opening distance D_{opening1} from each other in the opening position P_{opening}. The first opening distance D_{opening1} is arranged to be greater than the first initial distance Dᵢₙᵢₜᵢₐₗ₁. In addition, the first 112 and second 122 distal ends of the first 110 and second 120 arms of the first cuff opening assembly 100 are at a second opening distance D_{opening2} from the first 212 and second 222 distal ends of the first 210 and second 220 arms of the second cuff opening assembly 200, respectively, in the opening position P_{opening}. The second opening distance D_{opening2} is arranged to be greater than the second initial distance Dᵢₙᵢₜᵢₐₗ₂. Furthermore, the first opening distance D_{opening1} and the second opening distance D_{opening2} are arranged to be large enough to expand the cuff 22 of the glove 20 in the donning position P_{donning}. In other worlds, the first 112 and second 222 distal ends of the first 110 and second 120 arms of the first cuff opening assembly 100 and the first 212 and second 222 distal ends of the first 210 and second 220 arms of the second cuff opening assembly 200 in the opening position P_{opening} defines an opening area A_{opening}, where the opening area A_{opening} is greater than the area of the opening of the glove Ag in its relaxed state, shown in FIG. 8a.

As described above and illustrated in FIGS. 8a-b, the first 112, 212 and second 122, 222 distal ends are separated from each other when the first 110, 210 and second 120, 220 arms are rotated around the first GA1 and second GA2 geometrical axes, respectively, from the initial position Pᵢₙᵢₜᵢₐₗ towards the opening position P_{opening}. According to embodiments of the invention, the separation of the first 112, 212 and second 122, 222 distal ends during the rotation is achieved by arranging the first GA1 and second GA2 geometrical axes with an angle α to each other, i.e. the first GA1 and second GA2 geometrical axes have an angle α relative to each other. The angle α is selected to be different/other than 0 or 180 degrees, such that the first GA1 and second GA2 geometrical axes do not have the same orientation. Thus, the first GA1 and second GA2 geometrical axes are oblique, i.e. non-parallel, to each other. The orientation of the first geometrical axis GA1 thus differs from the orientation of the second geometrical axis GA2, such that the first GA1 and second GA2 geometrical axes intersect, as shown e.g. in FIGS. 8c-d.

FIGS. 8c-d show embodiments for a first cuff opening assembly 100. In FIG. 8c, the first cuff opening assembly 100 is shown in the initial position Pᵢₙᵢₜᵢₐₗ, in two different side views and in a top view. In FIG. 8d, the first cuff opening assembly 100 is shown in the opening position P_{opening}, in two different side views and in a top view. In the embodiments shown in FIGS. 8c-d, the first GA1 and second GA2 geometrical axes are arranged with an angle α to each other. The angle α is selected such that the first 112 and second 122 distal ends of the first cuff opening assembly 100 are at the first initial distance Dᵢₙᵢₜᵢₐₗ₁ from each other in the initial position Pᵢₙᵢₜᵢₐₗ, as shown in FIG. 8c. The angle α is further selected such that the first 112 and second 122 distal ends of the first cuff opening assembly 100 are separated to the first opening distance D_{opening1} from each other in the opening position P_{opening}, as shown in FIG. 8d, when the first 110 and second 120 arms are rotated around the first GA1 and second GA2 geometrical axes, respectively. In the same way, the first GA1 and second GA2 geometrical axes around which the first 210 and second 220 arms of the second cuff opening assembly 200 rotates may be arranged to each other with an angle α, where the angle α is selected such that the first 212 and second 222 distal ends of the second cuff opening assembly 200 are at the first initial distance Dᵢₙᵢₜᵢₐₗ₁ from each other in the initial position Pᵢₙᵢₜᵢₐₗ, and are separated to the first opening distance D_{opening1} from each other in the opening position P_{opening} (not shown in Figs.).

The rotation of the first 110, 210 and second 120, 220 arms around the first GA1 and second GA2 geometrical axes, respectively, may be achieved in different ways. FIGS. 9a-b show an embodiment where the rotation of the first 110 and second 120 arms of the first cuff opening assembly 100 takes place around a first 101 and a second 102 shaft, respectively. The first shaft 101 extends along the first geometrical axis GA1, while the second shaft 102 extends along the geometrical axis GA2. Furthermore, the first 101 and second 102 shafts are fixed in relation to each other, and possibly also in relation to the apparatus 800. The first arm 110 is arranged to rotate around the first shaft 101 extending along the first geometrical axis GA1 and the second arm 120 is arranged to rotate around the second shaft 102 extending along the geometrical axis GA2. In the embodiment shown in FIGS. 9a-b, the first arm 110 is rotatably attached to the first shaft 101 with a first attachment means 104, and the second arm 120 is rotatably attached to the second haft 102 with a second attachment means 106. The first 104 and second 106 attachment means may include e.g. a tubular sleeve or a similar arrangement.

In the embodiment shown in FIGS. 9b-c the rotation of the first 110 and second 120 arms of the first cuff opening assembly 100 is also based on a first 101 shaft extending along the first geometrical axis GA1 and a second shaft 102 extending along the geometrical axis GA2. However, in the embodiment shown in FIGS. 9b-c, the first 101 and second 102 shafts are instead arranged to rotate around their own axis, while maintaining the angle α relative to each other. Thus, in FIGS. 9c-d the first arm 110 is arranged to rotate together with the rotatable first shaft 101 extending along the first geometrical axis GA1 and being rotatable around the first geometrical axis GA1. Furthermore, the second arm 120 is arranged to rotate together with a rotatable second shaft 102 extending along the second geometrical axis GA2 and being rotatable around the second geometrical axis GA2. The first 101 and second 102 shafts may e.g. be attached to each other with a suitable coupling, which may include at least one gearwheel/cogwheel, e.g. a cardan/universal/gear joint. Although not shown in the figures, the rotation of the first 210 and second 220 arms of the second cuff opening assembly 200 may be achieved in the same way as for the embodiment shown in FIGS. 9a-b or as for the embodiment shown in FIGS. 9c-d for the first cuff opening assembly 100.

According to some embodiments of the invention, each of the first arms 110, 210 and the second arms 120, 220 of the first 100 and second 200 cuff opening assemblies, respectively, comprise two parts resiliently connected and arranged at an angle to each other. In this way, the insertion of the first arms 110, 210 and the second arms 120, 220 of the first 100 and second 200 cuff opening assemblies, respectively, into the cuff 22 of the glove 20 may be simplified. In such embodiments, the first arm 110, 210 comprises a first proximal part 113, 213 and a first distal part 114, 214 resiliently connected to each other. Furthermore, the second arm 120, 220 comprises a second proximal part 123, 223 and a second distal part 124, 224 resiliently connected to each other. The first 113, 213 and the second 123, 223 proximal parts may e.g. be resiliently connected to the first 114, 214 and the second 124, 224 distal parts, respectively, by means of at least one spring joint or a resilient material of the first 110, 210 and second 120, 220 arms.

FIGS. 10a-b show a top view of a first 100 and a second 200 cuff opening assembly where the first 110, 210 and second 120, 220 arms of the first 100 and second 200 cuff opening assemblies, respectively, comprise two resiliently connected parts. As FIGS. 10a-b shows a top view only the second arms 120, 220 of the first 100 and second 200 cuff opening assemblies, respectively, are visible. The second arm 120 of the first cuff opening assembly 100 comprises a second proximal part 123 and a second distal part 124 resiliently connected with a spring joint 107. Furthermore, the second arm 220 of the second cuff opening assembly 200 comprises a second proximal part 223 and a second distal part 224 resiliently connected with a spring joint 207. Although not shown in FIGS. 10a-b, the first 110, 210 arms of the first 100 and second 200 cuff opening assemblies are in a similar way comprising a first proximal part 113, 213 and a first distal part 114, 214 resiliently connected to each other, e.g. with spring joints.

In FIG. 10a the second arms 120, 220 of the first 100 and second 200 cuff opening assemblies, respectively, are shown in the initial position Pᵢₙᵢₜᵢₐₗ. In the initial position Pᵢₙᵢₜᵢₐₗ, the first 114 and second 124 distal parts of the first cuff opening assembly 100 may be in contact with the first 214 and second 224 distal parts of the second cuff opening assembly 200, respectively. Thereby, the first distal part 114 of the first cuff opening assembly 100 are at a first initial angle βᵢₙᵢₜ₁ to the first proximal part 113 of the first cuff opening assembly 100, and the first distal part 214 of the second cuff opening assembly 200 are at a third initial angle βᵢₙᵢₜ₃ to the first proximal part 213 of the second cuff opening assembly 200 (not shown in the Figs.). Furthermore, the second distal part 124 of the first cuff opening assembly 100 are at a second initial angle βᵢₙᵢₜ₂ to the second proximal part 123 of the first cuff opening assembly 100, and the second distal part 224 of the second cuff opening assembly 200 are at a fourth initial angle βᵢₙᵢₜ₄ to the second proximal part 223 of the second cuff opening assembly 200, as shown in FIG. 10a.

In the embodiment shown in FIG. 10a, the length of the second distal parts 124, 224 and the second proximal part 123, 223 are selected such that the second distal part 124 of the first cuff opening assembly 100 and the second distal part 224 of the second cuff opening assembly 200 are parallel to each other and in contact with each other in the initial position Pᵢₙᵢₜᵢₐₗ. Thereby, the second initial angle βᵢₙᵢₜ₂ and the fourth initial angle βᵢₙᵢₜ₄ is approximately 90 degrees in the initial position Pᵢₙᵢₜᵢₐₗ, as shown in FIG. 10a. In a similar way, the length of the first distal parts 114, 214 and the first proximal part 113, 213 are selected such that the first distal part 114 of the first cuff opening assembly 100 and the first distal part 214 of the second cuff opening assembly 200 are parallel to each other and in contact with each other in the initial position Pᵢₙᵢₜᵢₐₗ. Thereby, the first initial angle βᵢₙᵢₜ₁ and the third initial angle βᵢₙᵢₜ₃ is approximately 90 degrees in the initial position Pᵢₙᵢₜᵢₐₗ (not shown in Figs.).

When the first 110, 210 and second 120, 220 arms rotate around the first GA1 and second GA2 geometrical axes, respectively, the first 114 and second 124 distal parts of the first cuff opening assembly 100 will move away from the first 214 and second 224 distal parts of the second cuff opening assembly 200, leaving room for each distal part to move relative to the proximal part it is connected to. Due to the resilient connection between each distal and proximal part, the rotation may hence lead to that the angle β between the distal parts and the proximal parts changes, typically increases. Furthermore, as the first 110, 210 and second 120, 220 arms rotate, the first 114, 214 and second 124, 224 distal parts of the first 100 and second 200 cuff opening assemblies meet an inside of the cuff 22 of the glove 20 and starts to expand the cuff 22 of the glove 20. The resistance from the glove 20 leads to a force on the first 114, 214 and second 124, 224 distal parts of the first 100 and second 200 cuff opening assemblies, which will further increase the angle β between the distal parts and the proximal parts.

FIG. 10b shows the second arms 120, 220 of the first 100 and second 200 cuff opening assemblies, respectively, in different subsequent position as the second arms 120, 220 of the first 100 and second 200 cuff opening assemblies rotate away from the initial position Pᵢₙᵢₜᵢₐₗ towards the opening position P_{opening}. As shown in FIG. 10b, the angle β between the second distal part 124 and the second proximal part 123 of the first cuff opening assembly 100 increases when the second arm 120 of the first cuff opening assembly 100 rotates away from the initial position Pᵢₙᵢₜᵢₐₗ. In a similar way, the angle β between the second distal part 224 and the second proximal part 223 of the second cuff opening assembly 200 increases when the second arm 220 of the second cuff opening assembly 200 rotates away from the initial position Pᵢₙᵢₜᵢₐₗ. The angle β between the second distal part 124 and the second proximal part 123 of the second arm 120 of the first cuff opening assembly 100 in a subsequent position is herein denoted a second subsequent angle β_{sub2}, and the angle β between the second distal part 224 and the second proximal part 223 of the second arm 220 of the second cuff opening assembly 200 in a subsequent position is herein denoted a fourth subsequent angle β_{sub4}, as shown in FIG. 10b. Corresponding subsequent angles for the first arms 110, 210 of the first 100 and second 200 cuff opening assemblies are denoted a first subsequent angle β_{sub1} and a third subsequent angle β_{sub3}, respectively.

When the second arms 120, 220 of the first 100 and second 200 cuff opening assemblies, respectively, have reached the opening position P_{opening}, the second subsequent angle β_{sub2} and the fourth subsequent angle β_{sub4} may be approximately 180 degrees, as shown in FIG. 10b.

FIG. 10b shows the subsequent position for the second arms 120, 220 of the first 100 and second 200 cuff opening assemblies as they rotate towards the opening position P_{opening}. However, also the first arms 110, 210 of the first 100 and second 200 cuff opening assemblies behave in a similar way. Consequently, in a subsequent position, the first 114, 214 and second 124, 224 distal parts of the first 100 and second 200 cuff opening assemblies are in contact with an inside of the cuff 22 of the glove 20, such that the first distal part 114 of the first cuff opening assembly 100 is at a first subsequent angle β_{sub1} to the first proximal part 113 of the first cuff opening assembly 100, the second distal part 124 of the first cuff opening assembly 100 is at a second subsequent angle β_{sub2} to the second proximal part 123 of the first cuff opening assembly 100, the first distal part 214 or the second cuff opening assembly 200 are at a third subsequent angle β_{sub3} to the first proximal part 213 of the second cuff opening assembly 200, and the second distal part 224 of the second cuff opening assembly 200 are at a fourth subsequent angle β_{sub4} to the second proximal part 223 of the second cuff opening assembly 200.

The first subsequent angle β_{sub1} is greater than the first initial angle βᵢₙᵢₜ₁, the second subsequent angle β_{sub2} is greater than the second initial angle βᵢₙᵢₜ₂, the third subsequent angle β_{sub3} is greater than the third initial angle βᵢₙᵢₜ₃, and the fourth subsequent angle β_{sub4} is greater than the fourth initial angle βᵢₙᵢₜ₄. Furthermore, when the subsequent position corresponds to the opening position P_{opening}, the first subsequent angle β_{sub1}, the second subsequent angle β_{sub2}, the third subsequent angle β_{sub3}, and the fourth subsequent angle β_{sub4} are approximately 180 degrees.

The rotation of the first 110, 210 and second 120, 220 arms of the first 100 and second 200 cuff opening assemblies around the first GA1 and second GA2 geometrical axes may be provided in several different ways. According to some embodiments of the invention at least one cuff opening arrangement 600 further comprises at least one rotation providing means coupled to at least one of the first 110, 210 and second 120, 220 arms of the first 100 and second 200 cuff opening assemblies. The rotation providing means may be arranged to cause the rotation of at least one of the first 110, 210 and second 120, 220 arms of the first 100 and second 200 cuff opening assemblies between the initial position Pᵢₙᵢₜᵢₐₗ and the opening position P_{opening}. Furthermore, the first 110 and second 120 arms of the first cuff opening assembly 100 may be coupled such that the rotation providing means causes the first 110 and second 120 arms to rotate together, i.e. at the same time and in the same rotation direction, around or together with the first 101 and second 102 shafts, respectively. In a similar way, the first 210 and second 220 arms of the second cuff opening assembly may be coupled such that the rotation providing means causes the first 210 and second 220 arms to rotates together, i.e. at the same time and in the same rotation direction, around or together with the first 201 and second 202 shafts, respectively.

According to embodiments of the invention the rotation providing means may comprises a mechanical means, such as e.g. at least one rod displaced by a piston, or electrical means such as e.g. rotary engines.

It is to be noted that although some embodiments of the invention are herein described for a first cuff opening assembly 100 or a first 100 and a second 200 cuff opening assembly, e.g. the embodiments describe with reference to FIGS. 8a-10b, the same applies to any cuff opening assembly used in one or more cuff opening arrangements 600 of the apparatus 800.

1. An apparatus arranged for facilitating donning a garment comprising a cuff on a part of a human body, said garment initially being included in a package stored in a storage, comprising:
a fetching arrangement arranged for fetching said package from said storage, and for placing said package in a package opening position, said fetching arrangement including:
an arm arranged movable between said storage and said package opening position;
at least one holding means arranged on said arm for holding said package at a first end portion of said package being opposite to a second end portion where said cuff of said garment is to be extracted from said package;
a garment and package opening arrangement arranged for moving a first and a second side of said package away from each other in said package opening position, whereby said package and said cuff of said garment are opened; and
a cuff opening arrangement arranged for receiving said opened cuff of said garment, and for further opening said cuff to facilitate said donning of said garment.

2. An apparatus according to claim 1, wherein said at least one holding means is further arranged for holding said package and said garment at said first end portion of said package, whereby said package and said garment are held essentially vertically in said package opening position with said second end portion facing away from said at least one holding means.

3. An apparatus according to claim 1, wherein said at least one holding means is further arranged for holding said package at said first end portion of said package, whereby said package and said garment are held essentially horizontally in said package opening position with said second end portion facing away from said at least one holding means.

4. An apparatus according to claim 1, wherein said at least one holding means includes one or more in the group of:
at least one clamp;
at least two pressing fingers;
at least two rollers.

5. An apparatus according to claim 1, wherein said fetching arrangement further includes at least one retrieving means arranged for retrieving said package from said storage before said at least one holding means grabs hold of at least said package at said first end portion.

6. An apparatus according to claim 5, wherein said retrieving means include one or more in the group of:
at least one clamp;
at least two pressing fingers;
at least one roller;
at least one suction device;
at least one adhesive covered surface.

7. An apparatus according to claim 1, wherein said arm is extendable and tiltable.

8. An apparatus according to claim 1, further including a package end opener arranged for opening said package at said second end portion.

9. An apparatus according to claim 8, wherein said package end opener includes one or more in the group of:
at least one cutting device;
at least one ripping device.

10. An apparatus according to claim 1, wherein said garment and package opening arrangement includes a first and a second grasping device arranged for grasping said first and second sides of the package, respectively.

11. An apparatus according to claim 10, wherein each one of said first and second grasping devices includes at least one in the group of:
at least one suction device;
at least one clamping device arranged for clamping one of said first and second sides of said package;
at least one clamping device arranged for clamping at least one protrusion arranged on one of said first and second sides of said package;
at least one adhesive surface device arranged for adhering to one of said first and second sides of said package.

12. An apparatus according to claim 10, wherein said first and second grasping devices are arranged to be moved away from each other when they have grasped said first and second sides of said package, respectively, thereby opening said package.

13. An apparatus according to claim 1, wherein when said package is opened by said first and second sides being moved away from each other, a space is created between said first and second sides of said package 30 into which said garment can relax, whereby said cuff is opened.

14. Apparatus according to claim 1, wherein said cuff opening arrangement is arranged for placing said further opened cuff in a donning position.

15. Apparatus according to claim 1, wherein said cuff opening arrangement is arranged for receiving said garment in said package opening position.

16-18. (canceled)
